(19) United States
(12) Patent Application Publication
(10) Pub. No.: US 2025/0207174 A1
(43) Pub. Date: Jun. 26, 2025

(54) TARGETED MASS SPECTROMETRY METHODOLOGY FOR DETECTING KLOTHO ISOFORMS

(71) Applicant: THE TRUSTEES OF INDIANA UNIVERSITY, Bloomington, IN (US)

(72) Inventors: Kenneth J. LIM, Whitestown, IN (US); Arvin HALIM, Indianapolis, IN (US); Gayatri NARAYANAN, Indianapolis, IN (US); Amber L. Mosley, Carmel, IN (US)

(21) Appl. No.: 18/921,482

(22) Filed: Oct. 21, 2024

Related U.S. Application Data
(60) Provisional application No. 63/592,848, filed on Oct. 24, 2023.

(51) Int. Cl. C12Q 1/40; C12Q 1/37; G01N 30/02; G01N 30/14; G01N 30/72; G01N 30/88

(52) U.S. Cl. CPC C12Q 1/40; C12Q 1/37; G01N 30/14; G01N 30/7233; G01N 2030/027; G01N 2030/8831; G01N 2333/924; G01N 2333/976

(57) ABSTRACT

Compositions and methods are disclosed for detecting isoforms of α-Klotho present in a biological sample obtained from a patient.

### CROSS REFERENCE TO RELATED APPLICATIONS

This application claims priority to U.S. Provisional Patent Application No. 63/592,848 filed on Oct. 24, 2023, the disclosure of which is expressly incorporated herein.

### STATEMENT OF GOVERNMENT SUPPORT

This invention was made with government support under CA082709 and TR002529 awarded by National Institutes of Health. The Government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: “413687 Sequence Listing.xml” created on Jan. 21, 2025 and 35 KB in size.

### BACKGROUND

Klotho is a transmembrane protein that, in addition to other effects, provides some control over the sensitivity of the organism to insulin and appears to be involved in ageing. There are three subfamilies of klotho: α-klotho (alpha-Klotho), β-klotho (beta-Klotho), and γ-klotho (gamma-Klotho). α-klotho functions as a co-factor for the phosphatonin FGF23, and β-klotho functions as a cofactor for metabolic endocrines FGF19 and FGF21.

α-Klotho is canonically expressed as a single-pass transmembrane protein, which can be cleaved by proteases (ADAM10/17, BACE1) into soluble isoforms: soluble KL1 (sKL1)+soluble KL2 (sKL2) and soluble full-length ectodomain α-Klotho (sFL-K) (See FIG. 1). Formation of sKL1 and sKL2 requires simultaneous cleavage of α-Klotho at both its α1-cut site and α2/β-cut site, whereas sFL-K requires cleavage only at the α1-cut site. α-Klotho may also undergo alternative splicing of its mRNA transcript resulting in two additional potential isoforms: secreted KL1 (secKL1) and secreted full-length α-Klotho (secFL-K). The protein form of secFL-K has yet to be confirmed in humans. There are also α-Klotho isoforms with similar structures and sequences but may differ in a few amino acids (e.g., KL-VS variant) that also have clinical significance.

There currently exists no robust, quantitative assay that can distinguish between biologically active, soluble and secreted isoforms of α-Klotho in biological samples such as blood, urine and cerebrospinal fluid (CSF). α-Klotho is an anti-aging protein that is predominantly produced in the kidneys. Total circulating α-Klotho levels decline with aging and this decline is associated with age-related diseases such as heart failure, vascular calcification, chronic kidney disease, and neurodegenerative disorders. Replenishment or supplementation with certain isoforms of exogenous α-Klotho have been shown to be protective against the progression of age-related diseases in animal models. Measuring α-Klotho in serum can therefore be integral in assessing health status of a patient that is or is not undergoing treatments.

Accordingly, α-Klotho protein is gaining widespread pharmaceutical attention due to its therapeutic properties for the treatment of multiple conditions (reversal of aging, cancer, cardiovascular disease, kidney failure, neurologic conditions, arthritis). Multiple clinical studies have shown evidence supporting the use of α-Klotho as a predictive biomarker to gauge the risk of and to assess progression of multiple diseases (such as those listed above). However, current commercial assays (by ELISA for example) are unable to distinguish between the various isoforms of α-Klotho due to limitations in antibody-based techniques. Accordingly, there is a need for an assay that is capable of distinguishing the various isoforms to better understand their roles in disease progression and provide novel therapeutic strategies.

As disclosed herein a novel assay is provided for detecting α-Klotho isoforms, including for example the KL1 isoform. The assay of the present disclosure is based on targeted proteomic data and mass spectrometry analysis.

### SUMMARY

Current assays (ELISA, IP-IB, mass spectrometry) for α-Klotho do not distinguish between the separate isoforms of α-Klotho within a single sample, and often vary in their quantification of α-Klotho. Most assays predominantly focus on targeting the KL1 domain of α-Klotho, but this domain is shared between multiple isoforms (see FIG. 1). The isoforms of α-Klotho likely exhibit different physiological effects predominantly depending on if the isoforms contain KL1 or KL2 domains (or segments) or the combined KL1 and KL2 ectodomain. The level of these isoforms may have implications regarding the health/disease state of a patient. Several studies show that the level of total serum/circulating α-Klotho has been significantly associated with several aging-related diseases (e.g., metabolic syndrome, cardiovascular disease, kidney failure). Given this, distinction and quantification of α-Klotho isoforms are anticipated to be of high clinical significance/interest. Specifically, selected protein isoforms of α-Klotho, including soluble KL1 (sKL1), soluble KL2 (sKL2), secreted Klotho/KL1 (secKL1) and soluble ectodomain Klotho (sFL-k) are of current interest as there is current evidence supporting a differential role for each of these isoforms. Additionally, and most importantly, multiple animal studies have shown that treatment with sKL1 and secKL have beneficial effects (e.g., cardioprotective, enhance cognition).

In accordance with one embodiment of the present disclosure, a novel targeted mass spectrometry-based assay is provided that detects and quantifies different isoforms of α-Klotho, including, but not limited to, full-length ectodomain of α-Klotho, KL1 and KL2 domains of α-Klotho, and alternatively spliced secreted α-Klotho in biological samples, including but not limited to blood, cerebral spinal fluid, and urine. In accordance with one embodiment, a method is provided for detecting and quantifying α-Klotho isoforms in plasma or serum.

In one embodiment α-Klotho and its soluble isoforms, including the full-length ectodomain and the KL1 and KL2 domains, and the secreted isoforms, are detected in a serum sample by first depleting high abundant serum proteins and subjecting the proteins to enzymatic digestion, followed by targeted mass spectrometry coupled with liquid chromatography-tandem mass spectrometry (LC-MS/MS)-based parallel reaction monitoring (PRM) assay.

In one embodiment, the serum samples are depleted of major serum proteins using a commercially available serum depletion kit. Any of the known standard strategies for depletion of the highest abundant proteins in serum can be used in conjunction with the present methods, including kits that rely on multiple-use HPLC columns or multiple-use spin columns. Once the step of abundant protein depletion has been completed, the serum-depleted sample are subjected to chymotrypsin digestion to form peptides of α-Klotho, which are then analyzed and quantified by a targeted parallel reaction monitoring (PRM) liquid chromatography-tandem mass spectrometry (LC-MS/MS) assay. Briefly, peptides are ionized by electrospray ionization (ESI) to form parental ions, isolated using a mass to charge (m/z) inclusion list, fragmented using collisional dissociation (HCD or CID) and precursor and fragment ions are utilized to quantify α-Klotho isoform-specific peptides using standard peptide quantitation curves.

### BRIEF DESCRIPTION OF DRAWINGS

FIG. 1: provides a schematic representation of α-Klotho structure, how its various isoforms are produced, and their location in vivo.

FIGS. 2A-2C provide schematic representations of the general scheme used to detect circulating (soluble or secreted) α-Klotho isoforms in human (FIG. 2A), mouse (FIG. 2B), and rat (FIG. 2C) species.

FIG. 3 is a schematic of the LC-MS/MS process used to identify α-Klotho isomers.

FIG. 4 illustrates a table that describes the detectable peptides that identify each α-Klotho isoform within each species (human, mouse, rat).

### DETAILED DESCRIPTION

### Definitions

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide that has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

“Subject” refers to any mammal for whom diagnosis, treatment, or therapy is desired including mammals, e.g., humans, laboratory animals (e.g., primates, rats, mice, rabbits and guinea pigs), livestock (e.g., cows, sheep, goats, and pigs), household pets (e.g., dogs, cats, and rodents), and horses.

“Biological sample” refers to any tissue or fluid sample that comprises proteins including but not limited to blood, cerebral spinal fluid, saliva, serum, and urine.

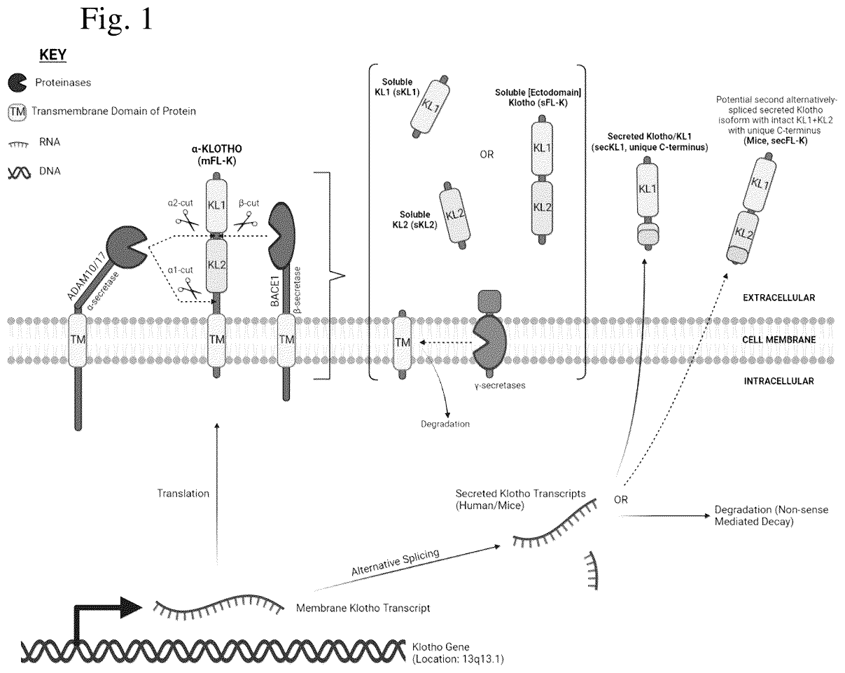

FIG. 1: provides a schematic representation of α-Klotho structure, how its various isoforms are produced, and their location in vivo. The α-Klotho gene is located on chromosome 13 in humans, chromosome 5 in mice, and chromosome 12 in rats. Transcription of the α-Klotho gene results in an mRNA transcript that contains 5 exons and will be translated into a single-pass transmembrane protein (mFL-K). The mFL-K's structure includes an N-terminal signal sequence, the KL1 domain, KL1-KL2 interdomain linker containing α2/β-cut site, the KL2 domain, KL2-transmembrane domain linker containing the α1-cut site, the transmembrane domain, and a short intracellular C-terminus segment. The mFL-K isoform can be cleaved at α1-cut site by ADAM10/17 to produce sFL-K. Additionally, mFL-K can also be cleaved simultaneously at both the α2/β-cut site by ADAM10/17 or BACE1 and at the α1-cut site to produce sKL1 and sKL2. The α-Klotho transcript may undergo further processing by alternative splicing before translation to yield two different secreted isoforms that are not bound to the cell membrane, secKL1 and secFL-K.

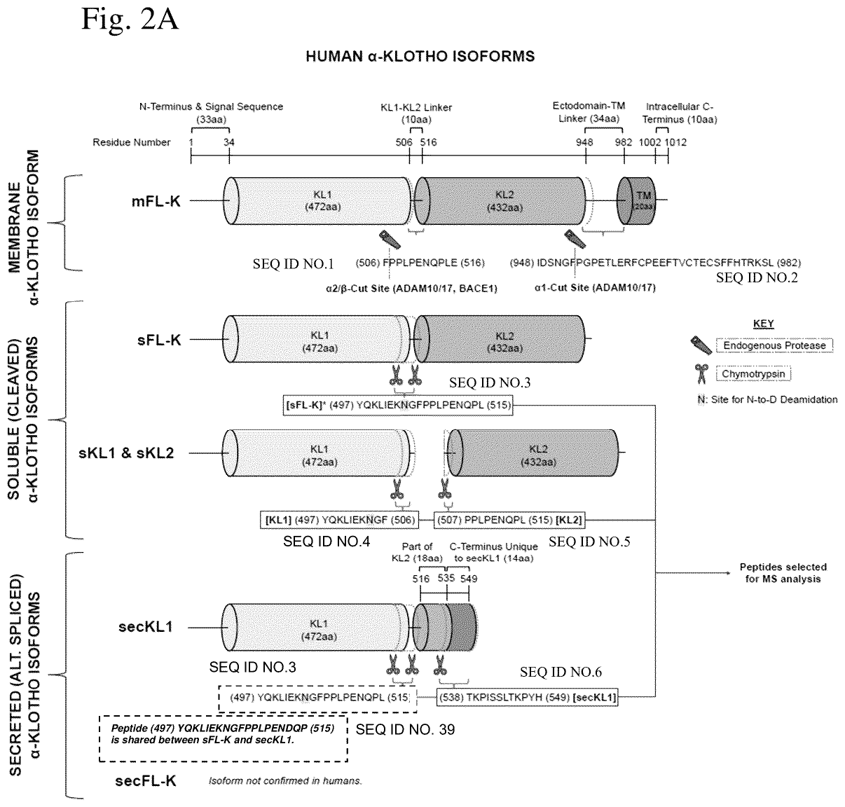

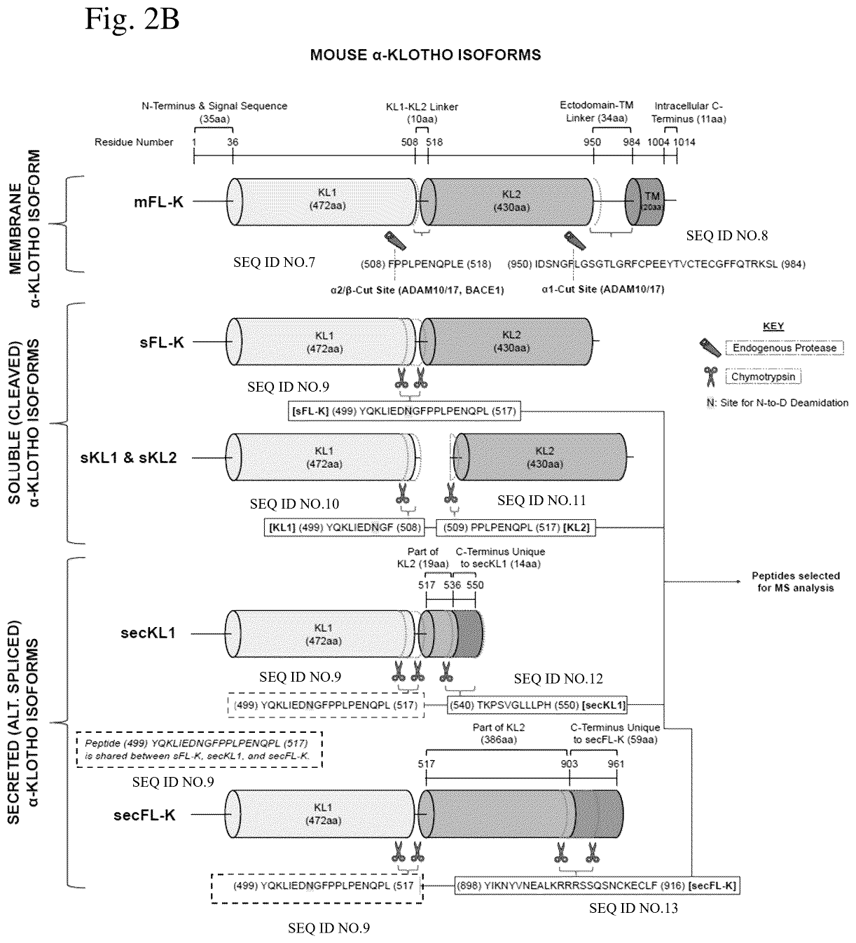

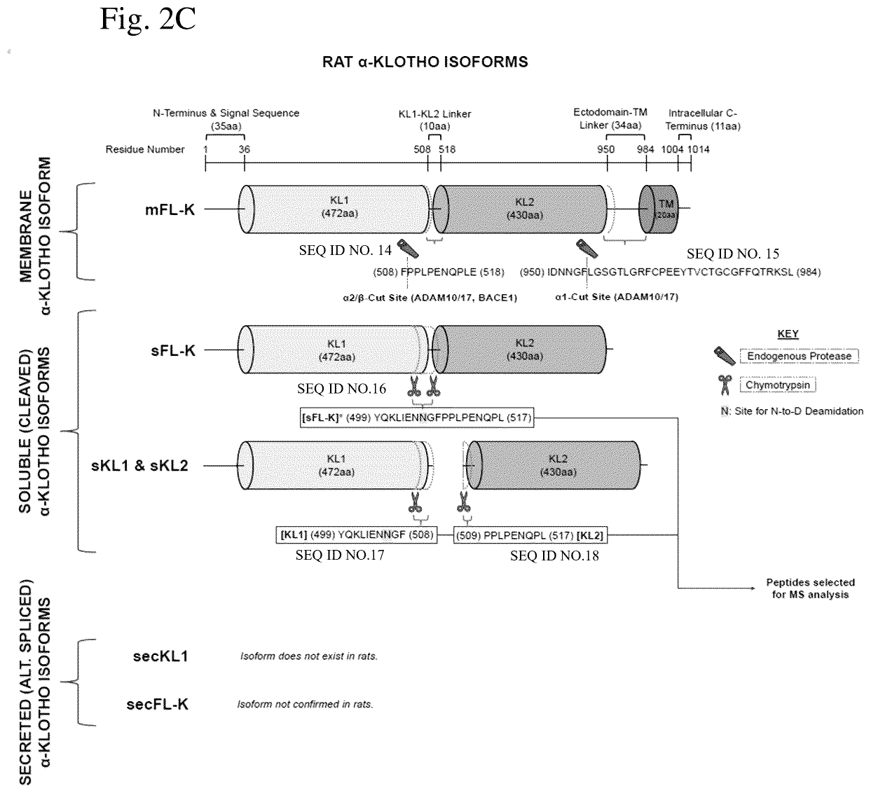

FIGS. 2A-2C provide schematic representations of the general scheme used to detect circulating (soluble or secreted) α-Klotho isoforms in human (FIG. 2A), mouse (FIG. 2B), and rat (FIG. 2C) species. Chymotrypsin digestion of the isoforms results in small α-Klotho peptides. Peptides to be used to quantify each α-Klotho isoform were selected based on their sequence and length that is unique to or best identifies their respective isoform (solid outline). The peptide sequences identifying the sFL-K isoform are shared with secKL1 and secFL-K isoforms (dashed outline) in the species having both isoforms. The secFL-K isoform has not yet been confirmed in humans.

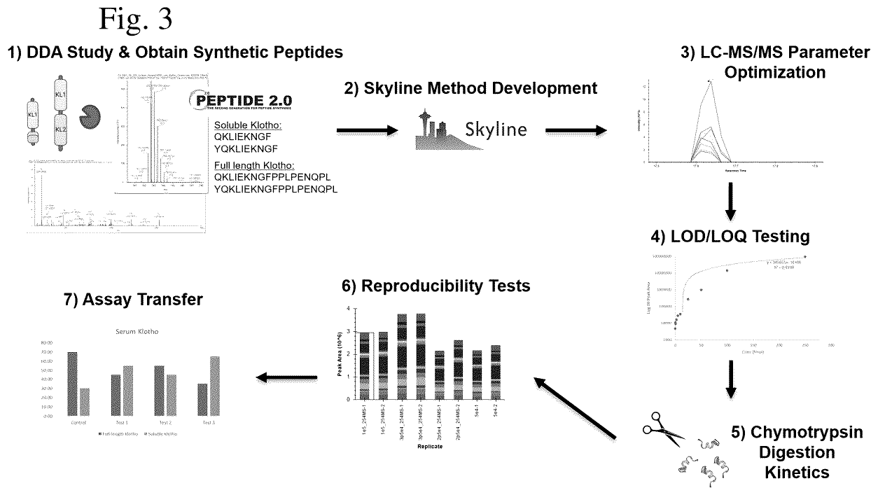

FIG. 3 is a schematic of the LC-MS/MS process used to identify α-Klotho isomers. Data-dependent acquisition (DDA) for selecting peptides of interest are performed by digestion of standard recombinant proteins of α-Klotho isoforms and analysis via LC-MS/MS. Synthetic peptides are obtained from the sequences found from the results of the DDA study and are used for parameter optimization and spike-in controls. Skyline targeted mass spec tool is an open-source program that helps analyze peptide ion spectra results from LC-MS/MS. LOD/LOQ refers to limit of detection and limit of quantification which is the lowest levels of the α-Klotho isoform peptides required to be detected by LC-MS/MS. Chymotrypsin digestion kinetics is a study to find optimal amount of and incubation time of chymotrypsin with the biological sample of interest to effectively digest α-Klotho proteins within the sample. Reproducibility tests are performed to ensure robustness of the assay, and assay transfer involves comparison with currently available α-Klotho assays.

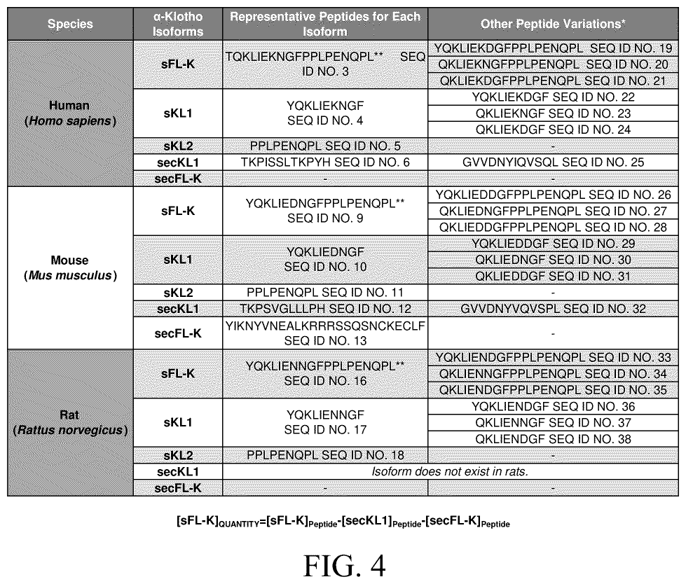

FIG. 4 illustrates a table that describes the detectable peptides that identify each α-Klotho isoform within each species (human, mouse, rat). *Variations of the peptide signatures that identify each α-Klotho isoform can occur as a byproduct of enzymatic digestion or by an endogenous or artificial modification. The sum of all variations detected equals the total level of the corresponding α-Klotho isoform. **All variations of the peptide identifying the sFL-K isoform are shared with the secKL1 and secFL-K isoforms. Therefore, the peptide signature selected to identify sFL-K will also identify secKL1 and secFL-K. A mathematical equation is provided below for calculating total sFL-K concentrations with known concentrations of secKL1 and secFL-K, which are identified by separate, unique peptide signatures. The total sFL-K concentration is determined by subtracting the total concentration of peptides unique to both secKL1 and secFL-K isoforms from the total concentration of the shared peptide signature that identifies the sFL-K, secKL1 and secFL-K isoforms.

$${[ s F L - K ]}_{Q U A N T I T Y} = {[ s F L - K ]}_{P E P T I D E} - {[ secKL 1 ]}_{P E P T I D E} - {[ secFL - K ]}_{P E P T I D E}$$

### EMBODIMENTS

The present methodology is based on bottom-up proteomic analysis where all proteins extracted from a sample are enzymatically digested together (rather than purified separately as is done in top-down proteomics). The resulting peptides are separated by liquid chromatography (LC) and then processed by mass spectrometry (MS). This is also considered as “shotgun proteomics”. The resulting peptide mass spectra (i.e., charge vs. mass) are compared to pre-specified sequence database and matched with proteins of interest. The bottom-up proteomic approach allows for efficiency and ease of assessing clinical biological samples without the need for an additional protein extraction step.

In accordance with one embodiment, parallel reaction monitoring (PRM) is conducted using a Q-Orbitrap mass analyzer to monitor/track sets of peptide ions, mass-charge (m/z) ratios and all fragment ions created from this inclusion within the same method. This is in comparison to selected reaction monitoring (SRM) which specifically monitors for a specified peptide ion and a specified fragment ion. PRM allows for a multi-peptide analysis in a single run, therefore a more comprehensive assay development with higher throughput.

In accordance with one embodiment, the present invention is directed to a targeted detection procedure that allows for the identification of unique sequences associated with the specific α-Klotho isoforms to be detected. In particular, in silico studies were used to find putative enzymes to digest α-Klotho proteins that would result in desired peptide sequence/region of interest. More particularly, commercially-available recombinant Klotho proteins (sKL-F: Recombinant Human Klotho (aa 34-981) Protein Carrier-Free, R&D Systems (Cat. No. 5334-KL) and secKL: Recombinant Human Klotho, PeproTech (Cat. No. 100-53) were used as model standards for α-Klotho isoforms. α-Klotho proteins were subject to different enzymatic digestion (i.e., testing trypsin, chymotrypsin, AspN) and chymotrypsin was experimentally determined to provide the desired cleavage. Specifically, only chymotrypsin (versus regularly used trypsin) was found to digest α-Klotho to obtain a peptide that allows to differentiate between sFL-K, sKL1, and sKL2. After chymotrypsin digestion, the quantity and quality of peptides resulting from chymotrypsin digestion can be compared to synthetic peptide standards to determine the relative concentration of sFL-K, sKL1, and sKL2 and the ratio of sKL1, and sKL2 present in a patient's sample (blood, cerebral spinal fluid, or urine.

Chymotrypsin does not normally cleave after Phe (F, P1 site) with a Pro (P, P1′ site) residue after it. However, recent studies investigating the similar property (known as Keil Rule) for trypsin, showed that trypsin may cut some proteins with Pro at P1′ position. Accordingly, chymotrypsin may still cleave certain proteins that have Pro at the P1′ site. However, the double Pro of the Klotho α2/β-cut site (F−/−PP) may further prevent the possibility of cleavage at this site. It has been experimentally confirmed that chymotrypsin does not cleave at the α2/β-cut site of α-Klotho, thus validating the present approach for detecting the soluble isoforms of α-Klotho.

One aspect of the present invention is the identification of an ADAM10/17, BACE1 natural cleavage site (α2/β-cut) located in an amino acid sequence (YQKLIEKNGFPPLPENQPL) (SEQ ID. NO. 3) located between the KL1 and KL2 domains. It is shown that enzymatic cleavage using chymotrypsin may cleave the α-Klotho polypeptide to release the intact peptide fragment YQKLIEKNGFPPLPENQPL (SEQ ID. NO. 3). The detection of the fragments YQKLIEKNGF (SEQ ID. NO. 4) and PPLPENQPL (SEQ ID. NO. 5) reveals in vivo activity of ADAM10/17, BACE1 and the presence of sKL1 and sKL2 in the biological sample. Studies have shown that α2/β-cut can only occur simultaneously with the α1-cut or only when α-Klotho is in its membrane form (mFL-K). Therefore, sFL-K cannot be further cleaved into sKL1 and sKL2 while in circulation. Accordingly, the present method is the first to use chymotrypsin to fragment α-Klotho polypeptides recovered from a patient's biological sample and take advantage of the natural endogenous cleavage processing of α-Klotho to differentiate the different isoforms of α-Klotho.

In accordance with one embodiment, a method of detecting α-Klotho isoforms in a patient's biological sample is provided. The biological sample can be any tissue or fluid sample that comprises proteins. In one embodiment, the biological sample is a biological fluid selected from saliva, urine, blood or a blood component. In one embodiment, the biological sample is serum. In one embodiment the method comprises the steps of obtaining a biological sample from a patient, optionally wherein the sample is a serum sample; enzymatically treating said sample with chymotrypsin; conducting LC-MS/MS analysis on said enzymatically treated serum sample, and detecting the relative concentration of a soluble α-Klotho isoform selected from soluble KL1 (sKL1), soluble KL2 (sKL2), secreted isoform (secKL1) and soluble full-length ectodomain Klotho (sFL-K).

In one embodiment, the biological sample is subjected to a protein depletion step where high abundance proteins such as albumin and hemoglobin are removed from the biological sample prior to the step of cleaving with chymotrypsin. In one embodiment, the total sFL-K level in a biological sample is determined by subtracting the total sum quantity of peptides unique to both secKL1 and secFL-K isoforms from the total quantity of peptides that identify the sFL-K isoform. This mathematical deduction is required because the peptides designated to identify the sFL-K isoform are shared with the secKL1 and secFL-K isoforms.

### Example 1

### Bottom-Up Proteomics Development Workflow

### Step 1A: Data-Dependent Acquisition (DDA) Study

In silico studies were conducted to find putative enzymes to digest α-Klotho protein that would result in desired peptide sequence/region of interest.

Commercially-available recombinant α-Klotho proteins were used as model standards for α-Klotho isoforms undergo different enzymatic digestion (i.e., testing trypsin, chymotrypsin, AspN) to experimentally confirm enzyme selection (i.e., chymotrypsin). sKL-F: Recombinant Human Klotho (aa 34-981) Protein Carrier-Free, R&D Systems (Cat. No. 5334-KL); secKL: Recombinant Human Klotho, PeproTech (Cat. No. 100-53).

The quantity and quality of peptides resulting from chymotrypsin digestion was assessed.

### Step 1B: Synthetic Peptide Acquisition.

Synthetic peptides based on the results of Step 1A were obtained and used to run LC-MS/MS parameter optimization, LOD/LOQ experiments, and spike-in tests as controls.

### Step 2: Skyline Method Development

Open-source software (Skyline Targeted Mass Spec Environment) was used to analyze mass spectrometry data. The software was used for targeted method development.

### Step 3: LC-MS/MS Parameter Optimization

Optimize LC-MS/MS parameters was be developed for all Klotho peptides. Some parameters were optimized for all peptides. Some parameters were specific for each peptide. The parameters included, gradient, charge state, collision energy (CE), fragmentation technique. injection time, automatic gain control (AGC), and resolution. Gradient is the optimal solvent (or solution) that provides the best LC separation of peptides. Charge State is the selection of the most abundant charge state of each peptide ions that also result in the best fragmentation pattern. Collision Energy (CE) is the energy imparted upon precursor peptide ions against neutral gas molecules causing fragmentation of precursor peptide ions. Fragmentation Technique in the method to breakdown peptides into smaller peptide fragments. Injection Time is the time to fill Orbitrap with peptide fragment ions which is reflective of how fast ions enter the Orbitrap. Ion injection time is the rate-determining step of mass analysis. Automatic Gain Control (AGC) is the threshold of how many peptide fragment ions to accumulate prior to peptide ion entry into Orbitrap. Resolution is the smallest difference between spectral peaks of different mass-to-charge (m/z) ratios that can be distinguished as separate spectral peaks.

### Step 4: Limit of Detection/Quantification (LOD/LOQ)

Limit of Detection (LOD) represents the lowest concentration of analyte that can be detected with acceptable signal-to-noise ratio. Limit of Quantitation (LOQ) represents the lowest concentration of analyte that can be quantified with repeatability/high confidence. Multiple analyte injections/testing was conducted to determine limits of MS detection and quantification based on each Klotho peptide to be detected. Synthetic peptides in either neutral or biological (serum) matrix background was tested.

### Step 5: Chymotrypsin Digestion Kinetics

Digestion of proteins by chymotrypsin in different biological matrices (with or without serum depletion) and incubation times was conducted to identify optimal conditions for producing an array of detectable protein fragments that allow for the identification of different isoforms of α-Klotho, including full-length ectodomain of α-Klotho, KL1 and KL2 domains of α-Klotho, and alternatively spliced secreted α-Klotho in biological samples.

### Step 6: Reproducibility Tests

The optimal conditions were repeated to assess reproducibility/consistency of results.

### Step 7: Assay Transfer

The LC-MS/MS method was applied in comparison to current standard assays.

### Comparison to Other Assays:

### Immuno-Biological Laboratories, Inc. (IBL-America):

Human Soluble Alpha Klotho ELISA (SKU 27998) is an antibody-based assay used to measure KL1-containing isoforms in serum, plasma, urine, and cell culture lysates. This is the most commonly used assay in literature and does not differentiate between KL1-containing isoforms. Unlike the LC-MS/MS method, this antibody-based assay cannot measure KL2, effectively measuring total soluble α-Klotho.

Human Secreted Alpha Klotho ELSIA (SKU 27901) is an antibody-based assay to measure secKL1 by its unique C-terminus in serum, plasma, and cerebrospinal fluid. This is a newly released assay. This antibody-based assay may have potential conflict with published/public antibody detecting the same isoform.

### ThermoFisher Scientific, University of Texas Southwestern

Targeted Selective Reaction Monitoring (SRM), Surface-Induced Dissociation Mass Spec Assay is a triple quadrupole MS assay measuring primarily KL1 domain peptides and one KL2 domain peptide. This assay requires AQUA heavy isotope peptide standards and trypsin digestion. Based on peptides selected for this assay, this assay would not be able to distinguish different types of α-Klotho isoforms.

1. A method of detecting α-Klotho isoforms in a patient's biological sample, method comprising
providing a biological sample from the patient;
enzymatically treating the biological sample with chymotrypsin;
conducting liquid chromatography-tandem mass spectrometry (LC-MS/MS) analysis on the enzymatically treated biological sample, and
detecting the relative concentration of a soluble α-Klotho isomer selected from the group consisting of soluble KL1 (sKL1), soluble KL2 (sKL2), secreted isoform (secKL1) and soluble full-length ectodomain Klotho (sFL-K).

2. The method of claim 1, wherein the biological sample is selected from the group consisting of urine, saliva, plasma or serum.

3. The method of claim 1, wherein the biological sample is a serum sample.

4. The method of claim 1, further comprising a step of subjecting the biological sample to a protein depletion step to reduce the concentration of abundant serum proteins present prior to the step of enzymatically treating the biological sample.

5. The method of claim 4, wherein the biological sample is a serum sample and the protein depletion step comprises reducing the concentration of albumin.

6. The method of claim 4, wherein the biological sample is a serum sample and the protein depletion step comprises reducing the concentration of albumin and hemoglobin.

7. The method of claim 1, wherein the LC-MS/MS analysis step comprises the use of peptide ion monitoring (PRM) conducted using a Q-Orbitrap mass analyzer.

8. A method of detecting α-Klotho isoforms in a patient's biological sample comprising:
providing a biological sample from the patient;
enzymatically treating the biological sample with chymotrypsin;
conducting liquid chromatography-tandem mass spectrometry (LC-MS/MS) analysis on the enzymatically treated biological sample, and
detecting the relative concentration of more than one α-Klotho isoforms.

9. The method of claim 8, wherein the more than one α-Klotho isoforms is selected from the group consisting of soluble KL1 (sKL1), soluble KL2 (sKL2), secreted isoform (secKL1) and soluble full-length ectodomain Klotho (sFL-K).

10. The method of claim 8, wherein the biological sample is selected from the group consisting of urine, saliva, plasma or serum.

11. The method of claim 8, wherein the biological sample is a serum sample.

12. The method of claim 11, further comprising the step of subjecting the serum sample to a protein depletion step to reduce the concentration of abundant serum proteins prior to the step of enzymatically treating the biological sample.

13. The method of claim 12, wherein the biological sample is a serum sample and the protein depletion step comprises reducing the concentration of albumin.

14. The method of claim 12, wherein the biological sample is a serum sample and the protein depletion step comprises reducing the concentration of albumin and hemoglobin.

15. The method of claim 8, wherein the LC-MS/MS analysis comprises the use of peptide ion monitoring (PRM) conducted using a Q-Orbitrap mass analyzer.

16. A method of detecting α-Klotho isoforms in a patient's biological sample comprising:
enzymatically treating the biological sample;
conducting liquid chromatography-tandem mass spectrometry (LC-MS/MS) analysis on the enzymatically treated biological sample, and
detecting the relative concentration of a soluble α-Klotho isomer selected from the group consisting of soluble KL1 (sKL1), soluble KL2 (sKL2), secreted isoform (secKL1) and soluble full-length ectodomain Klotho (sFL-K).

17. The method of claim 16, wherein enzymatically treating the biological sample comprises treating the biological sample with chymotrypsin.

18. The method of claim 16, wherein the biological sample is selected from the group consisting of urine, saliva, plasma or serum.

19. The method of claim 16, further comprising the step of subjecting the biological sample to a protein depletion step to reduce the concentration of abundant serum proteins prior to the step of enzymatically treating the biological sample.

20. The method of claim 19, wherein the biological sample is a serum sample and the protein depletion step comprises reducing the concentration of albumin and hemoglobin.


SEQUENCE LISTING (39 sequences)

SEQ ID NO: 1 - protein, 11 aa, Homo sapiens
FPPLPENQPLE

SEQ ID NO: 2 - protein, 35 aa, Homo sapiens
IDSNGPPGPETLERFCPEEFTVCTECSFFHTRKSL

SEQ ID NO: 3 - protein, 19 aa, Homo sapiens
YQKLIEKNGFPPLPENQPL

SEQ ID NO: 4 - protein, 10 aa, Homo sapiens
YQKLIEKNGF

SEQ ID NO: 5 - protein, 9 aa, Homo sapiens
PPLPENQPL

SEQ ID NO: 6 - protein, 12 aa, Homo sapiens
TKPISSLTKPYH

SEQ ID NO: 7 - protein, 11 aa, Mus musculus
FPPLPENQPLE

SEQ ID NO: 8 - protein, 35 aa, Mus musculus
IDSNGPLGSGTLGTFCPEEYTVCTECGFFQTRKSL

SEQ ID NO: 9 - protein, 19 aa, Mus musculus
YQKLIEDNGFPPLPENQPL

SEQ ID NO: 10 - protein, 10 aa, Mus musculus
YQKLIEDNGF

SEQ ID NO: 11 - protein, 9 aa, Mus musculus
PPLPENQPL

SEQ ID NO: 12 - protein, 11 aa, Mus musculus
TKPSVGLLLPH

SEQ ID NO: 13 - protein, 25 aa, Mus musculus
YIKNYVNEALKRRRSSQSNCKECLF

SEQ ID NO: 14 - protein, 11 aa, Rattus norvegicus
FPPLPENQPLE

SEQ ID NO: 15 - protein, 35 aa, Rattus norvegicus
IDNNGPLGSGTLGRFCPEEYTVCTGCGFFQTRKSL

SEQ ID NO: 16 - protein, 19 aa, Rattus norvegicus
YQKLIENNGFPPLPENQPL

SEQ ID NO: 17 - protein, 10 aa, Rattus norvegicus
YQKLIENNGF

SEQ ID NO: 18 - protein, 9 aa, Rattus norvegicus
PPLPENQPL

SEQ ID NO: 19 - protein, 19 aa, Homo sapiens
YQKLIEKDGFPPLPENQPL

SEQ ID NO: 20 - protein, 18 aa, Homo sapiens
QKLIEKNGFPPLPENQPL

SEQ ID NO: 21 - protein, 18 aa, Homo sapiens
QKLIEKDGFPPLPENQPL

SEQ ID NO: 22 - protein, 10 aa, Homo sapiens
YQKLIEKDGF

SEQ ID NO: 23 - protein, 9 aa, Homo sapiens
QKLIEKNGF

SEQ ID NO: 24 - protein, 9 aa, Homo sapiens
QKLIEKDGF

SEQ ID NO: 25 - protein, 12 aa, Homo sapiens
GVVDNYIQVSQL

SEQ ID NO: 26 - protein, 19 aa, Mus musculus
YQKLIEDDGFPPLPENQPL

SEQ ID NO: 27 - protein, 18 aa, Mus musculus
QKLIEDNGFPPLPENQPL

SEQ ID NO: 28 - protein, 18 aa, Mus musculus
QKLIEDDGFPPLPENQPL

SEQ ID NO: 29 - protein, 10 aa, Mus musculus
YQKLIEDDGF

SEQ ID NO: 30 - protein, 9 aa, Mus musculus
QKLIEDNGF

SEQ ID NO: 31 - protein, 9 aa, Mus musculus
QKLIEDDGF

SEQ ID NO: 32 - protein, 12 aa, Mus musculus
GVVDNYVQVSPL

SEQ ID NO: 33 - protein, 19 aa, Rattus norvegicus
YQKLIENDGFPPLPENQPL

SEQ ID NO: 34 - protein, 18 aa, Rattus norvegicus
QKLIENNGFPPLPENQPL

SEQ ID NO: 35 - protein, 18 aa, Rattus norvegicus
QKLIENDGFPPLPENQPL

SEQ ID NO: 36 - protein, 10 aa, Rattus norvegicus
YQKLIENDGF

SEQ ID NO: 37 - protein, 9 aa, Rattus norvegicus
QKLIENNGF

SEQ ID NO: 38 - protein, 9 aa, Rattus norvegicus
QKLIENDGF

SEQ ID NO: 39 - protein, 19 aa, Homo sapiens
YQKLIEKNGFPPLPENDQP